(19) United States
(12) Patent Application Publication
(10) Pub. No.: US 2003/0100997 A1
(43) Pub. Date: May 29, 2003

(54) Matrix assays in genomically indexed cells

(76) Inventors: Damien J. Dunnington, San Diego, CA (US); Steven J. Brown, San Diego, CA (US); Pandi Veerapandian, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/139,068

(22) Filed: May 2, 2002

Related U.S. Application Data
(60) Provisional application No. 60/288,966, filed on May 4, 2001.

(51) Int. Cl. G06G 7/48; G06G 7/58; G06F 19/00; G01N 33/48; G01N 33/50

(52) U.S. Cl. 702/19; 703/11

(57) ABSTRACT

A method for ascertaining the functional patterns of pharmacologically-important compounds by measuring the physiological effect of a plurality of compounds on a plurality of cells comprising the steps of: assaying the plurality of compounds to obtain a first set of data determining the physiological effect of each compound on each cell; assaying at least one known pharmaceutically-important compound to obtain a second set of data determining the physiological effect of the known pharmaceutically-important compound on each cell; and comparing the first and second sets of data to identify a compound having similar physiological effects as the known pharmaceutically-important compound thereby ascertaining its functional patterns.

### RELATED APPLICATIONS

This application claims priority to U.S. Provisional Application Ser. No. 60/288,966, filed on May 4, 2001 and entitled MATRIX ASSAYS IN GENOMICALLY INDEXED CELLS. The entire contents of the provisional application are hereby incorporated by reference.

### TECHNICAL FIELD

The present invention relates to a method for identifying pharmaceutically important compounds by measuring the biological response of a library of cells in the presence of a compound. In particular, the present invention relates to ascertaining the biological activity profile of a compound by measuring the resulting physiological changes of at least one cell from a library of cell in a number of biological activity assays.

### BACKGROUND OF THE INVENTION

Discovery of new drug candidates is essential to combat various diseases. However, current drug discovery techniques are time consuming and expensive. These discovery methods commonly require a target such as an enzyme or receptor linked to a disease process, a means of measuring the function of the target, a source of chemical compounds, and a means of processing large quantities of data. Biologically active compounds identified using these methods are further tested to determine their functional activity, including selectivity for the desired target and organ, in whole cells and in vivo. Functionally active compounds are then tested for their adsorption, distribution, metabolism, elimination, and toxicology (“ADMET”) properties. Compounds that have both biological and functional activity as well as favorable ADMET values are then assessed for large-scale production and formulation.

Targets have also been frequently identified using genomic techniques such as studying the gene expression patterns in different tissue types. However, once identified the target must be validated or linked to a disease process. Validation occurs by genetically “knocking out” or overexpressing the gene of interest, by examining the function of the gene in a lower organism such as yeast or flies, or by demonstrating inhibition or activation after adding specific compounds, for example, cytokines known to be involved in disease processes.

Once a target is validated, a primary assay is developed enabling the testing of a number of chemical compounds. Typically, this assay expresses the gene of interest in a host organism that does not normally produce the gene product. The resulting protein is harvested, purified and tested directly. Alternatively, gene expression is determined indirectly by the detection of a measurable signal or a phenotypic change. Compounds are then evaluated for their effect in the assay. The source of compounds may be random collections, biased libraries that are structurally related to known modulators of the target, or compounds specifically designed or selected by using the molecular structure of the target.

Compounds found to be active in the primary assay are tested in secondary assays to ensure their activity is recapitulated in a more physiological setting. These are generally functional assays, which involve measuring the function of the target in its endogenous milieu in the presence of the compound. These assays are typically designed to mimic the disease process. For example, production of cytokines in response to allergens can be measured to identify potential therapeutic agents. Selectivity may also be determined by testing the compound against one or more related targets such as T_{H1} and T_{H2} profiling. Alternatively, compounds may be tested against unrelated targets using standard assay panels such as beta-adrenergic receptors to ensure lack of cardiovascular effects.

Compounds with the desired properties in the secondary assays are tested for ADMET properties. In parallel with these studies, new compound libraries are synthesized based on the characteristics of the compounds with the desired properties. These libraries are tested through the same secondary and ADMET assays to select those with the best combination of properties.

Compounds with favorable ADMET properties are tested in animal models of the disease under study. As part of the testing, the compounds are evaluated for in vivo drug metabolism and pharmacokinetics (“DMPK”), efficacy against the modeled disease process and adverse effects. Based on these results, candidate compounds are selected for large-scale synthesis and drug formulation studies. Following scale-up, candidates are evaluated in a more extensive pathology-toxicology studies from which development compounds are selected for Phase 1 safety studies in humans.

One of the major disadvantages of current drug discovery methods is that a potential drug candidate could be overlooked if it fails a single test. For example, a compound having structural features that contribute to good ADMET properties may be discarded because the compound demonstrated poor efficacy against the disease target. Similarly, lack of selectivity may mask new indications for a compound, especially if the number of secondary assays is limited. Because the target product profile of most drugs requires a combination of efficacy, selectivity, and ADMET properties, identification of a compound using current drug discovery methods is time consuming, resource intensive, and highly inefficient. In practice, less that 10% of active compounds identified from these methods will progress to the clinic, and many of these will fail during clinical trials. This inefficiency can be attributed partly to the linearity of testing one compound at a time and the design of current discovery methods.

Inefficiency can also be attributed to the segregation of disciplines. For example, genomics and structural chemistry have made great advances with their fields, but they remain separate entities. There is currently no effective way to link patterns of gene expression to functional activities by a compound's structure, other than by utilizing the methods described. Advances in structural biology will help in the construction of molecular models of proteins, against which compounds may be evaluated for possible testing against specific targets. However, such molecular modeling cannot recapitulate the multiple interactions occurring within a cell, many of which may be time or disease dependent. Similarly, chemical compounds frequently affect more than one biomolecule simultaneously and the methods that only look for a single activity often fail to detect potentially damaging side effects.

Therefore, there is a need for an improved process for drug discovery that can identify pharmacologically important compounds based on chemical structure, biological activities, gene expression, functional activity and tissue selectivity that is time efficient and cost effective.

### DESCRIPTION OF THE FIGURES

FIG. 1 is a photograph showing expression levels of the serotonin type 4 receptor in 31 cell lines. Each cell line was analyzed by rt-PCR using primers specific for the 5HT4 receptor and for the housekeeping genes beta actin and transferrin receptor. Control samples tested in the absence of reverse transcriptase are also shown.

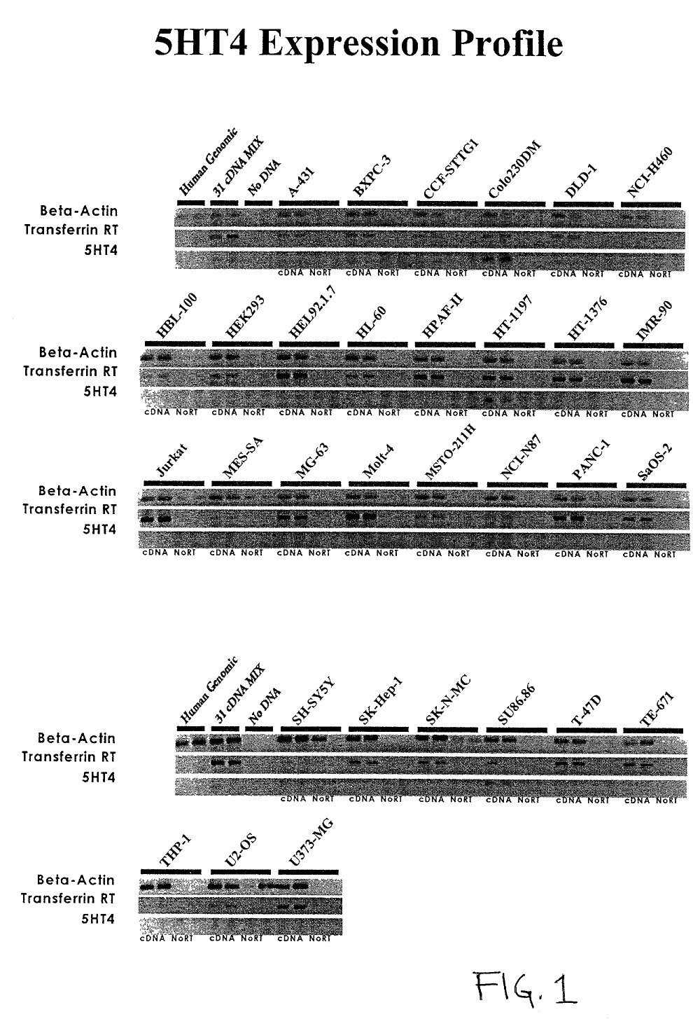

FIG. 2 is a bar graph showing cyclic AMP levels in 6 cell lines that express detectable levels of the 5HT4 receptor, after treatment with 10 uM cisapride. Control samples treated with solvent are also shown. Cyclic AMP levels are expressed as fmol per well.

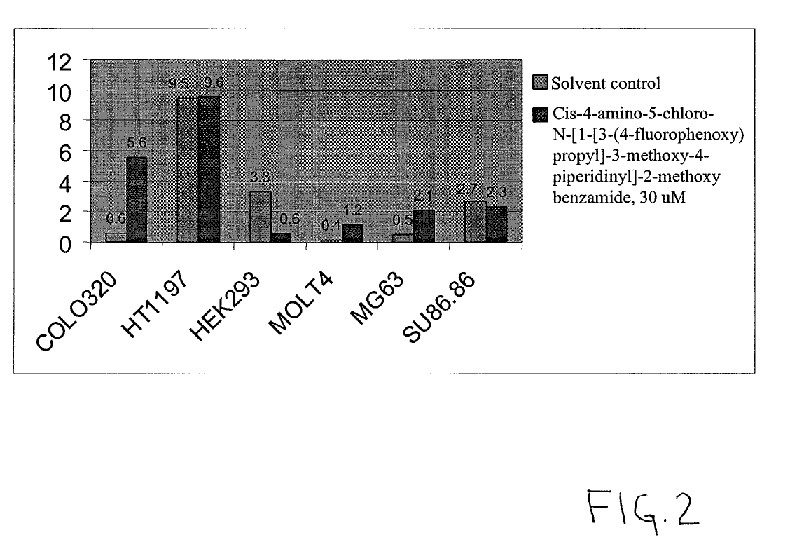

FIG. 3 is a photograph showing expression of histamine receptor subtypes 1 through 4 in 28 cell lines. Each cell line was analyzed by rt-PCR using primers specific for the indicated receptor or for the housekeeping genes beta actin and transferrin receptor. Control samples tested in the absence of reverse transcriptase are also shown.

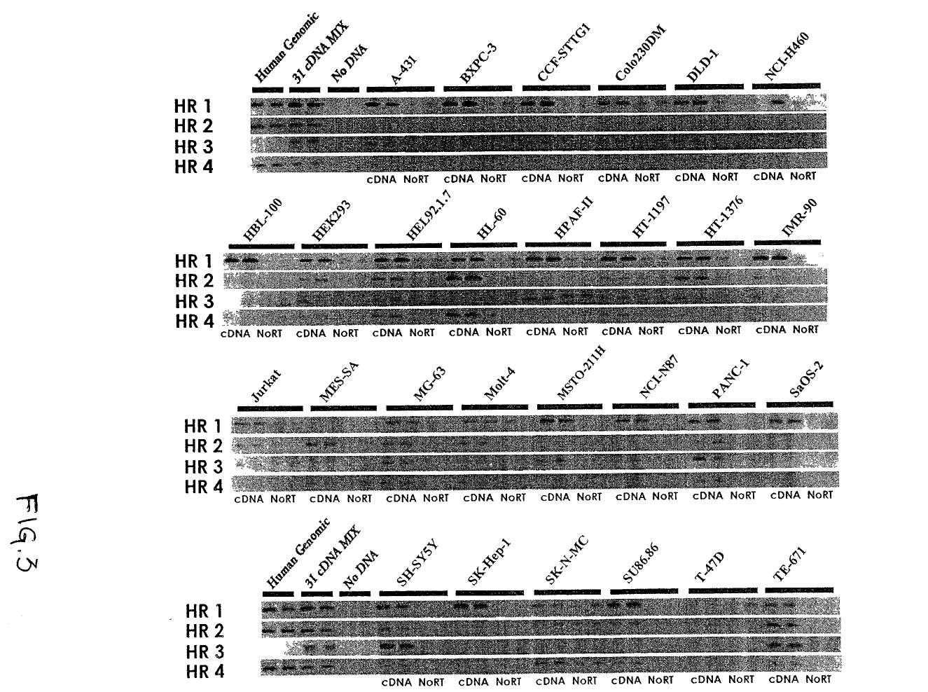

### SUMMARY OF THE INVENTION

In accordance with the present invention, a method for ascertaining the functional patterns of pharmacologically-important compounds by measuring the physiological effect of a plurality of compounds on a plurality of cells is disclosed comprising the steps of: assaying the plurality of compounds to obtain a first set of data reflecting the physiological effect of each compound of the plurality of compounds on each cell of the plurality of cells; providing a second set of data reflecting the physiological effect of at least one known pharmaceutically-important compound on each cell of the plurality of cells; and comparing the first and second sets of data to identify a compound of the plurality of compounds having similarities to or differences from the at least one known pharmacologically-important compound, thereby ascertaining the functional patterns of the identified compound.

In one aspect of the present invention, a method for inferring the biological activity of an uncharacterized compound by determining its physiological effect in a plurality of cells is disclosed comprising the steps of: assaying the uncharacterized compound to obtain a first set of data reflecting the physiological effect of the uncharacterized compound on each cell of the plurality of cells; providing a second set of data reflecting the physiological effect of the at least one compound of known biological activity on each cell of the plurality of cells; and comparing the first and second sets of data to determine similarities or differences between the physiological effects of the uncharacterized compound and the at least one compound of known biological activity, thereby inferring the biological activity of the uncharacterized compound.

In another aspect of the present invention, a method for selecting a compound, from a plurality of compounds, that has specificity for a target molecule, is disclosed comprising the steps of: assaying the plurality of compounds to obtain a first set of data reflecting the physiological effect of each compound on each cell of the plurality of cells; providing a second set of data reflecting which cells of the plurality of cells expresses the target molecule; and comparing the first and second sets of data to select a compound from the plurality of compounds that exhibit minimal effects on the cells of the plurality of cells that do not express the target molecule thereby selecting a compound having improved specificity for said target molecule.

In yet another aspect of the present invention, a method for identifying a compound, from a plurality of compounds, that modulates a target molecule, comprising the steps of: assaying the plurality of compounds to obtain a first set of data reflecting the physiological effect of each compound of the plurality of compounds on each cell of the plurality of cells; providing a second set of data reflecting which cells of the plurality of cells express or do not express the target molecule; assaying at least one known target molecule modulator to obtain a third set of data reflecting the physiological effect of the known target molecule modulator on each cell of the plurality of cells; and comparing the first, second and third sets of data to identify a compound having similar physiological effects as the known target molecule modulator.

In one embodiment of the present invention, the plurality of cells may be endothelial cells, connective tissue cells, epidermal cells, hemopoietic cells, stem cells or differentiated daughter cells derived from stem cells, central nervous system cells, endocrine cells, tracheobronchiolar cells muscle cells, urogenital cells or digestive tract cells. The endothelial cells may be atrial cells or vascular endothelial cells. The connective tissue cells may be osteoblast cells, osteoclast cells, chondrocyte cells, synoviocyte cells, fibrosarcoma cells, or osteocyte cells. The epidermal cells may be melanocyte cells, keratinocyte cells, skin fibroblasts cells, mammary ductal cells, mammary epithelial cells, corneal epithelial cells, hair follicle cells, papilla cells or submaxillary gland cells. The hemopoietic cells may be lymphoblast cells, monocyte cells, T-cells, B-cells, neutrophil cells, eosinophil cells, erythroblast cells, granulocyte cells or dendritic cells. The stem cells may be embryonic stem cells, teratocarcinoma cells, neural precursor cells or bone marrow stem cells. The central nervous cells may be astrocyte cells, ganglionic cells, cerebellum cells, neuroblast cells or neuronal differentiated cells. The endocrine cells may be pancreas cells, thyroid cells, pituitary cells, or adrenal cells. The tracheobronchial cells may be lung cells, tracheal cells, or bronchiolar epithelium cells. The muscle cells may be smooth muscle cells, striated muscles cells or cardiac muscle cells. The urogenital cells may be kidney epithelium cells, kidney mesangium cells, bladder cells, ovary cells, uterus cells, testis cells, placenta cells or prostate cells. The digestive tract cells may be liver cells, stomach cells, intestine cells, gall bladder cells, or esophagus cells.

In another embodiment of the present invention, the plurality of cells are healthy cells, diseased cells, or a combination of healthy and diseased cells. The diseased cells may be cells associated with a medical condition such as an infectious disease, cancer, an immune disease, a central nervous system disorder, a cardiovascular disease, a metabolic disorder, a musculoskeletal disorder, an epidermal disorder, a reproductive disorder or aging. The infectious disease cells may be virally infected cells, bacterially infected cells, fungally infected cells, protozoally-infected cells or mycobacterial infected cells. The cells associated with cancer may be carcinoma cells, sarcoma cells, mesothelioma cells, leukemia cells, melanoma cells, papilloma cells, glioblastoma cells, astrocytoma cells, neuroblastoma cells or metastatic tumor cells.

The medical condition may be an immune disease such as autoimmune disease cells, allergic disease cells, inflammatory disease cells, or immunodeficiency disease cells; a central nervous system disorder such as psychiatric disorder, a neurodegenerative disorder, a neuroinflammatory disorder, an affective disorder or a stroke; a cardiovascular disease such as hypertension, atherosclerosis, myocardial infarction, ventricular hypertrophy, cardiac arrhythmias, congestive heart failure or pulmonary hypertension; diabetes or obesity; a musculoskeletal disorder such as osteoarthritis, rheumatoid arthritis, osteoporosis or myasthenias; an epidermal disorder such as psoriasis, dermatitis, or alopecia; aging; or erectile dysfunction or infertility.

The carcinoma cells may be breast carcinoma cells, prostate carcinoma cells, ovarian carcinoma cells, non-small cell lung carcinoma cells, colorectal carcinoma cells or esophageal carcinoma cells. The virally infected cells may be cells infected with human immunodeficiency virus, cytomegalovirus, respiratory syncytial virus, rhinovirus, rotavirus, influenza virus, hantavirus or ebola virus. The plurality of cells may be a combination of healthy and diseased cells wherein the cells may be of the same histological origin, subclones of a parental cell, differentiated cells from a precursor cell population, or from a common tissue.

In yet another embodiment of the present invention, the physiological effects may be determined by assays for cellular membrane potential, intercellular calcium levels, cAMP levels, light scattering, gene expression, phenomenological assay, physiological transport, cell proliferation, physiological secretion, apoptosis or toxicity. The gene expression assay may be an assay that determines the production of disease-specific mRNAs or changes in cell surface markers. The phenomenological assay may be a morphology change assay, a temperature sensitivity assay, a motility assay, a syncytia formation assay, a chemotaxis assay or an adhesion assay. Examples of a physiological transport assay include a compound uptake assay or a compound efflux assay. The cell proliferation assay may be a DNA synthesis assay, an apoptosis assay or an anchorage-independent growth assay. Examples of a physiological secretion assay include a cytokine production assay, a hormone secretion assay or a neurotransmitter secretion assay. The toxicity assay may be a quantitative reactive oxygen species assay, an amyloid production assay, a mitochondrial membrane potential assay or a membrane integrity assay. The apoptosis assay may be a surface marker assay such as TNF receptor or annexin display, a DNA fragmentation assay such as staining with PCNA or an enzyme activity assay, such as a caspase activity assay.

In other aspects of the present invention, data structures comprising the sets of data obtained by the methods above are provided as well as a compound selected or identified utilizing any of the methods above.

Definitions

Prior to setting forth the invention, it may be helpful to first set forth the definitions of certain terms that will be used hereinafter. All references, which have been cited below are hereby incorporated by reference in their entirety.

The term “functional pattern” as used herein refers to a profile of a compound created from the physiological effects observed when a cell is placed in contact with the compound.

The term “pharmacologically-important compound” as used herein refers to a compound that may be useful in the treatment or prevention of a medical or diseased condition.

The term “physiological effect” as used herein refers to the changes a cell undergoes after exposure to a compound. Such changes may be observed using a variety of assays including for example a light scatter assay, a gene expression assay, a phenomenological assay, a physiological transport assay, a cell proliferation assay, a physiological secretion assay, an apoptosis assay, or a toxicity assay.

The term “biological activity” as used herein refers to the process of accomplishing an effect in a biological system. For example specific activity of an enzyme is the catalytic effect of exerted by an enzyme expressed as units per milligram of enzyme, or molecular activity of an enzyme which is the number of substrate molecules transformed per minute per molecule of enzyme.

The term “improved specificity” as used herein refers to the ability of a compound to affect an individual target without producing effects that are not related to the function of that target.

The term “minimal effects” as used herein refers to an insubstantial or an absence of a physiological effect when a cell is exposed to a compound.

The term “target molecule modulator” as used herein refers to an increase or decrease of a physiological effect resulting from the interaction of the compound with a particular cellular molecule of interest.

The term “data structure” as used herein refers to a collection of information on the physiological effect of a compound when placed in contact with a cell that may be correlated to identify a compound's biological activity.

### DETAILED DESCRIPTION OF THE INVENTION

The object of the present invention is to provide methods that may be used to develop a profile of a compound of interest based on the physiological effects in a plurality of cells exposed to the compound. Physiological effects are based on a number of parameters, including chemical structure, biological activities, gene expression and tissue selectivity.

In particular, methods are provided to ascertain the functional patterns or to infer the biological activity of pharmacologically-important compounds by measuring the physiological effect of a plurality of compounds on a plurality of cells comprising the steps of: assaying the plurality of compounds to obtain a first set of data determining the physiological effect of each compound on each cell; assaying at least one known pharmaceutically-important compound to obtain a second set of data determining the physiological effect of the known compound on each cell; and comparing the two data sets to identify a compound having similar physiological effects as the known compound thereby ascertaining the functional patterns or inferring the biological activity of the compound.

In addition, methods are provided that may be used to select or identify at least one compound from a plurality of compounds that modulates or that has improved specificity for a target molecule in at least one cell of a plurality of cells by assaying the plurality of compounds to obtain a first set of data determining the physiological effect of each compound on each cell; determining which cells of the plurality of cells expresses the target molecule to obtain a second set of data; assaying a known target molecule modulator to obtain a third set of data determining the physiological effect of the known target molecule modulator on each cell; and comparing the first, second and third sets of data to select or identify compounds having similar physiological effects as the known target molecule modulator and minimal effects on cells that do not express the target.

A compound profile is an accumulation of data representative of the physiological changes that result when a cell or cells is or are exposed to a compound. This profile is unique to each compound and based on the number of physiological changes monitored can form a virtual fingerprint of the functional activities of the compound with respect to a given cell type or family of cells. Assaying one or more known pharmaceutically active compounds may be used to identify critical fingerprint regions that may lead to the identification of other potential pharmaceutically important compounds. Comparison of the profiles of a compound with a set of known pharmacologically-important compounds can provide for an efficient and expeditious approach to identify other similar or more potent pharmacologically-important compounds. These methods for creating compound profiles remove limitations related to the linearity of current drug discovery methods that can eliminate potential candidates based on a single undesirable test result. In addition, a compound profile may be prepared for a given number of physiological effects known to be pharmacologically important to a specific medical condition thereby reducing and potentially eliminating compound failures that often result during the labor intensive and cost prohibitive process of current drug discovery methods.

I. Compounds

Profiles may be created for a variety of different compounds including for example, organic or inorganic molecules, nucleic acids or proteins. Compounds that may be profiled also include synthetic or naturally occurring compounds as well as portions of compounds such as nucleotides, oligonucleotides polynucleotides, polypeptides, or peptides. In addition, compounds may be altered to mimic a known pharmacologically-important compound or to modify particular activities of interest prior to being profiled. For example, a compound may be structurally altered to increase its half-life or decrease its immunogenicity in vivo. Particular characteristics of each of these classes of compounds may be considered when selecting compounds for assaying. Some of these characteristics may be universal for each class including for example structural configuration, biological activity, polarity, lipophilicity, dipole moment, molecular weight, as well as charge and charge density under particular conditions such as temperature, pressure, volume, concentration and pH. Other characteristics may be specific to particular classes such as for example, nucleic acids have the specific characteristics of comprising a sugar moiety (i.e., ribose or deoxyribose) and base sequence. In another example, the oxidation state of a complexed metal could be an important characteristic in the case of inorganic compounds.

Compounds may be grouped based on particular characteristics that have been shown, or are expected to have, desired physiological effects. For example, organic compounds having a steroidal four ring precursor configuration may be expected to have similar physiological effects on particular cells consequently these compounds may be grouped and tested together. If the compounds are grouped they may be tested simultaneously in an array that allows a large number of compounds to be assayed efficiently and expeditiously.

II. Cells

A wide variety of cells may be utilized with the present invention. The choice of cells selected will depend on the particular medical condition of interest. One skilled in the art will recognize that each type of cell may require a particular isolation procedure and culture condition for growth and proliferation. Isolation procedures and culture conditions can be found, for example, in Animal Cell Culture: A practical approach (J. R. Masters, Ed., Oxford University Press, third edition), 2000; Culture of Immortalized Cells (R. I. Freshney and M. G. Freshney, Eds., Wiley-Liss, Inc, New York, N.Y.), 1996; Harrison, M. A., and Rae, I. F., General Techniques of Cell Culture, Cambridge University Press, Cambridge, U.K. 1997; and Basic Cell Culture Protocols, (J. W. Pollard and J. M. Walker, Eds., Humana Press, Inc. Totowa, N.J., second edition), 1997. Cells and cell lines may also be purchased or obtained by collection services such as American Type Culture Collection (“ATCC”), Rockville, Md. Cells that are obtained from commercial sources may have specific and unique isolation and culture conditions that are often available or provided by the supplier.

Examples of cell types that may be used include but are not limited to atrial endothelial, vascular endothelial, osteoblast, osteoclast, chondrocyte, synoviocyte, fibrosarcoma, osteocyte, melanocyte, kertatinocyte, skin fibroblast, mammary ductal, mammary epithelial, corneal epithelial, hair follicle, papilla, submaxillary gland, lymphoblast, monocyte, T-cell, B-cell, neutrophil, eosinophil, erythroblast, granulocyte, dendritic, embryonic stem, teratocarcinoma, neural precursor, bone marrow stem, astrocyte, ganglionic, cerebellum, neuroblast, neuronal differentiated, pancreas, thyroid, pituitary, adrenal, lung, tracheal, bronchiolar epithelium, smooth muscle, striated muscle, cardiac muscle, kidney epithelium, kidney mesangium, bladder, ovary, uterus, testis, placenta, prostate, liver, stomach, intestine, gall bladder, esophagus, human immunodeficiency virus infected, cytomegalovirus infected, respiratory synctial virus infected, rhinovirus infected, rotavirus infected, influenza virus infected, hantavirus infected, ebola virus infected, bacterially infected, fungally infected, protozoally infected, mycobacterially infected, breast carcinoma, prostate carcinoma, ovarian carcinoma, non-small cell lung carcinoma, colorectal carcinoma, esophageal carcinoma, sarcoma, mesothelioma, leukemia, melanoma, papilloma, glioblastoma, astrocytoma, neuroblastoma, and metastatic cells.

Creation of cDNA Libraries of Cultured Cells

cDNA libraries may be created from cultured cells. Advantageously, prior to storage in array format, each cell line is grown to bulk culture and mRNA is extracted. The mRNA can either be stored directly as mRNA, or alternatively as cDNA. In a preferred embodiment, mRNA is reverse transcribed to its cDNA counterpart and stored as cDNA in order to maintain greater stability. cDNA samples are aliquoted in storage plates for PCR, or chemically labeled and stored for microarray analysis. For analysis of expression of specific genes, PCR primers are designed specifically to the target sequence(s) and used to amplify transcripts of the cDNA samples. For a more comprehensive analysis of the expression of a large number of genes, cDNA samples can be hybridized to microarrays of target probes, e.g., all known GPCRs. The association of a label with a specific probe may identify expression of that particular target by the cells. Such labels are well known to those of skill in the art. The patterns of gene expression can be analyzed to identify cell types that express specific targets of interest. This information can be correlated with functional data and tissue origin to identify functionally active receptors in anatomically distinct sites and link them to disease states or unexpected effects of compounds.

Storing Cells

Cells may be maintained in culture prior to assaying or may be frozen in an appropriate media for low temperature storage. When freezing cells, they may be prepared and frozen simultaneously or different cells may be prepared and frozen at different times. Cells may be stored in a storage container and may be grown in culture and transferred subsequently into an assay apparatus prior to assaying. This procedure may be desired when the cells are to be frozen for period of about 365 days or longer or when cells have a low viability in storage. A cell storage container may be any device that is able to accept cells in liquid culture, is preferable constructed of a material able to withstand −100° C. without sacrificing structural integrity, and may be autoclaved. A number of containers known to those skilled in the art may be used to store cultured cells such as for example flasks, plates, and tubes. When desired these containers may be made of materials that may be sterilized such as for example polystyrene and polypropylene (Fisher Scientific, Tustin, Calif.). Although individual cell lines or cell types may require unique storage conditions, many cells may be stored at a concentration from about 10² cells/mL to about 10⁸ cells/mL, preferably from about 1 cells/mL to about 1 cells/mL.

Storing Cells in an Assay Apparatus

Cells may also be aliquoted and stored directly in an assay apparatus. For example, the apparatus may be a biochip in which the assay is performed, or may be a cassette or multiwell plate which is inserted into an external instrument for reagent addition and/or detection of responses to compounds. Preferably the assay apparatus is constructed of a material able to withstand the temperature of cell storage without sacrificing structural integrity and in a configuration that allows multiple cell lines to be assayed simultaneously if desired. A preferred storage apparatus is a microtiter plate or silicon chip.

Positions within the assay apparatus may be reserved for newly prepared cells or assay controls that may be provided at a later in time.

III. Arrays

The methods of the present invention are preferably performed in an array format to allow quick and efficient collection of data. An array of cells may be prepared in a wide variety of configurations. These configurations may vary significantly, depending on the functional pattern being studied. A compound profile may be developed utilizing cells that are isolated from a single tissue or a variety of tissues, are involved in a diseased state, are representative of a disease's progression, possess a polymorphism, are isolated from different species, are isolated from various points during the aging process, or are isolated from a cloned organism.

Assemblage of a Representative Array of Human Cell Lines

Conventional tissue culture techniques do not have the capacity to allow simultaneous manipulation of hundreds of cell lines. Labor costs, time taken to grow large numbers of cell cultures, variable growth rates, and risks of cross-contamination make simple scaling of existing techniques prohibitive.

Much greater efficiency is achieved by testing hundreds of cells lines simultaneously consequently it would be beneficial to provide a plurality of human cell lines representative of most, and preferably all, tissues in the human body. Initially, efficiency is achieved by pre-formatting and storing cell arrays in assay-ready form. Cells can be grown by conventional tissue culture techniques and aliquoted in arrays. The arrays can either be multi-well plates, microchips, or the like. Each position in the array may contain a different cell line. Advantageously, containers are bar-coded and stored frozen. As additional cells become available, each plate or chip may be supplemented with new cells. This process can be automated, using techniques similar to those used for compound storage, and known by those skilled in the art.

The end result of the above process is a bank of plates containing a set of cell lines in array format. The contents of each plate and of the overall inventory can be stored in a database and accessed either through a workstation or via the bar code or other code (e.g., an RF chip)on the plate. It is possible to use mixed container storage, plates, or microchips for initial screening and vials for follow-up profiling. Multiple copies of each plate may advantageously be prepared and stored in several discrete freezers to protect against equipment failure. For example, if arrays are stored with cell lines in rows and different compounds in columns, a 384 well plate would have 16 cell lines in the rows and 24 compounds or controls in the columns. Alternatively, each cell line may be stored in a separate plate, allowing testing of compounds and controls in all 384 wells.

Alternatively, if flow-based readouts are used, cells can be individually labeled with specific markers and then several cell types can be mixed together. Containers with pre-coded mixtures of cells may be stored, frozen, and thawed for compound testing.

Assay Configurations

The selection of cells to be included within the assay apparatus may be based on a particular medical condition of interest or may be determined based on the assay procedures to be conducted. The configuration may be designed to assay a single cell line or cell type or may comprise a mixture of cell lines or cell types. When cells are mixed they are preferably chosen such that an assay result may be isolated for each cell line or cell type in the mixture as well as to avoid any interactions between the cells that would cause the assay results of any of the individual cell lines or cell types to vary from a result that would be obtained from assaying that cell line or cell type individually.

The cells may be organized in the assay apparatus in any configuration desired by the user. Preferably the configuration is designed to test a variety of cell lines or cell types against a variety of compounds as well as a variety of concentrations of compounds with a variety of dilutions of cell lines or cell types. For example, each row of an assay apparatus may correspond to a different cell line or dilution of a cell line and each column may represent the same cell line for screening dilutions of a single compound or for screening different compounds at a given dilution. Alternatively, rows may represent a single cell line while columns may correspond to dilutions of those cell lines. Cells may be aliquoted into the assay apparatus manually or by automation. Preferably the cells are aliquoted using automation to quickly and efficiently deposit the desired cell line or cell type in the desired concentration in the desired location within the assay apparatus. Simultaneous with the deposition of a cell line or cell type within the assay apparatus the cell type, concentration, dilution, and location within the apparatus are recorded. Preferably these data are collected and stored automatically within a computer accessible database. Alternatively, the user may record this information manually. This information may also be recorded directly on the assay apparatus by bar code or computer scanable or readable information chip.

Alternatively, two or more cell types may be individually tagged and mixed together for assaying with the test compound. Tagging may be accomplished by using transfected genetic markers or by staining the cells with colored or fluorescent dyes. In such cases, it is preferable that the detection instrumentation be capable of measuring responses from single cells. A preferred method of accomplishing this is to use flow cytometry. In this method, cells are tagged with fluorescent membrane dyes such as DiI, DiO and DiD (Molecular Probes, Eugene Oreg.), mixed together and exposed to the test compounds. For each cell in the mixture, simultaneous measurements can be made of tagging dye fluorescence and physiological response, using multi-color flow cytometry. Preferably, the data are analyzed by electronically gating physiological response signals according to the tag fluorescence, so that responses from the individual cell populations may be segregated.

A wide variety of configurations, or arrays may be created based on the desire of the user and the medical condition of interest. In particular, arrays may be prepared that are directed to a specific disease indication, specific tissue type or specific cell line.

Transfer of Stored Cells to Assay Apparatus

Cells that are stored may be transferred to an assay apparatus when desired by a variety of methods known to those skilled in the art. Generally cells are removed from storage, thawed, centrifuged, the freezing media replaced with fresh culture media and diluted to the desired concentration. The actual concentration of cells used can vary depending on the assay, however, cells are generally about 10⁴ cells/mL to about 10⁷ cells/mL, preferably from 10⁶ cells/mL to about 5×10⁶ cells/mL.

IV. Testing for Activity Using a Library of Chemical Compounds

When compounds are to be tested, the required number of plates are removed from storage and prepared as described above. A multichannel pipettor may be used to remove the freezing solution and add culture media. Deep well plates may be used for storage, and the cells may be aliquoted to several assay plates. For example, a conventional 96 or 384 well plate with each row containing a different cell line can be used for storage or as the assay plate. Thus, in this example, a 96 well plate can be used to test 11 compounds plus a control against 8 cell lines, and a 384 well plate can be used to test 23 compounds plus a control against 16 cell lines. Unexpectedly, multiple cell types were found cable of being frozen, stored, thawed, and immediately assayed in such an array format without loss of viability or response to the pharmacological agents.

Adherent cells can be seeded onto a matrix that can readily be dissolved by mild treatment protocols and assay plates are advantageously cultured for 2-3 days to allow the cells to recover. For example, collagen coated wells or beads can be used to allow adherent cells to attach. Cells can be released from the matrix by digestion with collagenase. It is important to use a release protocol that does not damage cell surface proteins. The cell suspension may be analyzed for responses to compounds, such as calcium mobilization, changes in cAMP levels or membrane potential. Preferably, such analysis is done using flow-based methods such as high throughput screen (HTPS) flow or high throughput flow cytometry (HT-FCM). In one method, cell suspensions are treated with a fluorescent detection reagent, combined in a flow stream with agonists, antagonists, or test compounds and the mixture is allowed to flow through a detector. In another method, cell suspensions are preincubated with agonists, antagonists or test compounds along with fluorescent detection reagents and introduced into a detector. Preferably the detector is a florescence-activated cell sorter (“FACS”) machine. The advantage of using FACS-based methods is that multiple readouts can be accommodated, and single cells are measured. Thus, simultaneous functional analysis of several cell types can be performed for each compound tested. Moreover, dead cells or subpopulations, e.g., in differentiating or primary cultures can be analyzed or excluded. Detailed information can be obtained in one step through multiplexing, resulting in higher information content and reduced turnaround time. Additionally, responding and non-responding cells may be physically separated without loss of viability and grown for further study, such as gene expression differences.

As an alternate method, a two-step process is possible in which initial screening is aimed at producing a yes/no answer. Any appropriate cellular signal can be used in this initial screening, including signals from G-protein coupled receptors, calcium, membrane potential, and cAMP readouts, which are used to detect compound activity. Conventional plate readers such as FLIPR or Tecan Ultra can be used in conjunction with calcium sensitive dyes or antibodies that bind cAMP. Screening may involve adding the compounds and looking for agonist effects, adding a known ligand to look for antagonist activity, and adding a standard such as a calcium ionophore or forskolin to induce a receptor-independent signal. The latter can be used to normalize responses to correct for variability in cell numbers or viability.

Once active compounds and responding cells are identified, cells may then be cherry-picked, either from the frozen store, or from a copy of the assay plate. Cells can be seeded into flasks, grown using standard tissue culture techniques, and tested using any appropriate technique, such as HTPS or HTPS-flow. (These techniques are disclosed in U.S. Pat. Nos. 6,096,501, 5,919,646, and 5,804,436, the entire disclosures of which are incorporated by this reference.) This gives detailed information about the mechanism of action, efficacy, and potency of the active compound.

V. Physiological Effects

The physiological effects that result from contacting a cell with a compound provide data to develop a compound profile that may be utilized to determine the pharmacological, therapeutic or diagnostic utility of the compound. The physiological effects may be obtained from a variety of assays available to those skilled in the art including for example light scattering assays, gene expression assays, phenomenological assays, protein activity assays, physiological transport assays, cell proliferation assays, physiological secretion assays or toxicity assays. The number and type of assays conducted may vary depending on the profiles desired. Correspondingly, the values obtained from these assays may be recorded in a variety of ways that provide ease of comparison to other collected data. For example, the data may be provided in raw form or may be converted into a desired unit of measure. Alternatively, an arbitrary number may be assigned to the assay result. For example, a value from one to ten may be assigned to the assay result, wherein the arbitrary number represents a range of assay values. Values may represent an increase or decrease of a physiological effect usually in comparison to a control or an amount of compound added to the assay to reach a desired physiological effect, and may further be normalized if desired. The preferred value is either an EC50 for agonists, a K₁ for antagonists/inhibitors, or a % control for readouts such as light scatter.

The analysis of the interactions between compound and cell may be different depending on the assay performed. The assay may directly measure the membrane features of the cell such as by flow cytometry or interaction may be inferred from secondary effects resulting from compound to cell binding. Some examples are calcium mobilization assay, changes in cAMP levels, or membrane potential. The assay may be a one-step-multiplexing assay or may be a two step method.

A one step-multiplexing assay is an assay that is able to detect multiple parameters. When the assay is a one step-multiplexing assay, it may be flow based. In the one step multiplexing flow based method, the cell suspension may be treated with a fluorescent detection reagent combined with agonists, antagonists, or test compound, and the mixture is streamed through a detector. Alternatively, the cell suspension may be preincubated with agonists, antagonists, or test compounds and then introduced into a detector along with fluorescent detection reagents. Preferably the detector is a flow cytometer that may be used to obtain multiple readings on single cells. Utilizing this system, dead cells or subpopulations of cells may be analyzed or excluded. Preferably the cytometer is equipped with cell sorting capabilities such that responding or non-responding cells may be isolated and cultured for further analysis such as gene expression differences. (Cytometry 43:211-6 (2001); Irving D., Am Clin Lab 16:16-7 (1997); Mann R C., Cytometry 8:184-9 (1987).

When the assay is a two-step process, the first step generally detects compound to cell interactions such as binding while the second step involves further study of the mechanism of action identifying the physiological effect of that interaction. For example, in the case of G-protein coupled receptors (“GPCRs”), the first step may include measurement of calcium levels, membrane potential, and cAMP levels. Screening may involve adding the compound and determining agonist effects, adding a known ligand and detecting antagonist activity, and adding a standard such as a calcium ionophore or forskolin to induce a receptor-independent signal.

Testing compounds for agonist activity may be accomplished by contacting each cell line with each compound and measuring a functional response such as calcium mobilization, cyclic AMP production or changes in membrane potential.

Testing compounds for antagonist activity may be accomplished by contacting each cell line with each compound and a known agonist or stimulant of the target(s) of interest or general targets of pharmaceutical importance. Functional responses such as calcium mobilization, cyclic AMP production or changes in membrane potential may then be measured.

It may be useful to compare the magnitude, kinetics, and qualitative features of responses of multiple cells to compounds that are known to specifically affect a target. Such agents could include native ligands for receptors or ion channels, cytokines, growth factors, chemokines, neurotransmitters, hormones pro-inflammatory peptides, or compounds known to specifically interfere with signaling by these agents.

Similarly, it may be useful to correlate the biological fingerprints of a compound with patterns of gene expression in the different cell types, tissues from which the cells originate and chemical structures of the compounds to provide information regarding the efficacy, selectivity, and unexpected effects of chemotypes and molecular targets that they affect.

Calcium levels may be assayed by adding and subsequently measuring calcium-sensitive dyes. cAMP levels may be measured by adding and subsequently measuring labeled antibodies or antibody fragments that bind cAMP. Both may be detected using conventional plate readers such as the FLIPR (Fluorescent Imaging Plate Reader Molecular Devices, Sunnyvale, Calif.) or Tecan Ultra (Tecan, Durham N.C.).

Once cell samples having positive results are identified, the corresponding cell samples are seeded into flasks and are grown and further assayed to determine the mechanism of action, efficacy and potency of the compound.

Gene expression may be correlated with functional data and tissue origin to identify functionally active receptors in anatomically distinct sites that may be linked to particular disease states or unexpected physiological effects of the compounds being assayed. The term “genomic expression” as used herein refers to a profile of expressed genes. Information regarding genomic expression may be obtained by exposing a compound to multiple cell lines or types and identifying the resulting modulation (such as, for example, activation or suppression) of a gene transcript.

To determine gene expression patterns of a cell, mRNA is extracted from the cell using any method known to those skilled in the art. Commercial kits may be used, such as those available from Qiagen Inc, Valencia Calif., or Clontech, Palo Alto Calif. The mRNA may then be reverse-transcribed into cDNA if desired. (Sambrook, Protocols in Molecular Biology). The mRNA or cDNA may be aliquoted for storage or analyzed by RT-PCR or hybridized microarray analysis to determine the gene expression of the cell. Id.

Analysis may show altered production of mRNA that corresponds to one or more cellular proteins. Expression is considered altered when production is increased or decreased, more particularly when the difference in production of a particular mRNA transcript is 100 copies per cell or greater, preferably 1 or greater copies per cell.

FLIPR Protocol

Cellular responses to compounds such as calcium mobilization or fluctuations in membrane potential or calcium channel migration may be evaluated using a conventional FLIPR. Cells can be aliquoted to wells of black, clear bottom poly-L-lysine 96 well plates treated and maintained overnight at 37° C., 5% CO₂ in a humidified incubator. Media is advantageously removed and the cells may then incubated in HBBS/10 mM HEPES pH 7.4 containing probenicid and Fluor-3AM (Molecular Probes, Eugene Oreg.) for one hour at 37%. Antagonists can be added to the indicated concentration and the cells incubated at room temperature for 20 minutes. After 10 seconds of baseline read collection, the agonists may be added on the FLPR and data may be collected for a total of 3.5 minutes. Preferably, the fluorescence emission is normalized to initial intensity and the sum of the signal is exported and used for determining percent of control responses using the following formula: Percent of Control=(Sample-unstimulated cells)/(ionomycin treated cells-unstimulated cells).

HTPS Protocol

Automated characterization of pharmaceutically important compounds and the measurement of their effects on various cells may be achieved via high-throughput pre-screening (HTPS) technology. For example, calcium channel activity may be measured by HTPS technology. Preferably, cells are fed fresh media the day before use and are grown to ˜80% confluence. On the day of the assay, cells may be harvested with 0.1% EDTA in PBS or 0.1% EDTA, 0.25% Trypsin in HBSS (Cellgrow, Herndon Va.) if necessary. Advantageously, the cells re washed with PBS and resuspended in Hybridoma media (Sigma, Chicago Ill.) at 2 million cells/ml containing 0.4 uM Fura-2AM (Molecular Probes, Eugene Oreg.) and incubated at 25° C. for 1 hour with gentle mixing. In the HTPS, the agonists and cells may be incubated for 30 seconds prior to entering the fluorometer flow cell. The change in intracellular calcium induced by agonist can be observed by monitoring Fura-2 fluorescence.

VI. Compound Profiles

Compound profiles can be developed based on the particular interest of the user and will dictate the configuration of the assay. This is particularly desirable when the compound has no known function because the assay has the potential to encompass a wider range of responses and disease applications unlike currently used drug discovery methods. For example, cells such as osteoclasts and osteoblasts isolated from connective tissue which provide bone maintenance functions may be useful in developing a compound profile relating to osteoporosis. In contrast, cells isolated from the epidermis may be useful in developing a compound profile relating to psoriasis.

Compound profiles may be generated using a single readout (such as calcium mobilization). Alternatively, compound profiles may be generated using a combination of readouts. For example, calcium, membrane potential and cyclic AMP would be useful for GPCRs.

A profile may comprise data collected from the observed physiological effects on healthy and diseased cells exposed to a compound of interest. By examining the differences in the observed physiological effects, a potential drug target may be identified that could lead to the development of a therapeutic candidate able to exploit those differences.

A profile may comprise data collected from observed physiological effects on cells isolated during the progression of a disease and exposed to a compound of interest. Preferably, the time points correspond to different stages of the disease including most preferably a non-diseased sample that predates the disease progression. By examining the differences in the observed physiological effects, a potential target may be identified that would be useful in detecting a change indicative to the onset or progression of the specific disease.

A profile may comprise data collected from cells obtained from multiple individuals at various points of disease progression and are designated according to stage of progression. This configuration may be desired when multiple samples are not available from the same patient.

A profile may comprise data collected from physiological effects on cells containing a polymorphism to a compound of interest. Polymorphisms are found in different geographical locations and between races often correlating to reduced or increased susceptibility to disease. By examining the differences in the observed physiological effects, a potential therapeutic candidate may be designed either mimicking or reducing the effect of the polymorphism.

In one embodiment, cells from a subject with a polymorphism that provides a benefit can be compared to those that do not possess the polymorphism. Differences in compound interaction may predict the form of a new therapeutic by mimicking the effect of the polymorphism. In another configuration, cells from a subject with a polymorphism that increase the likelihood of disease can be compared to one that does not possess the polymorphism. This configuration may be desired to develop drugs that treat those with polymorphisms.

A profile may comprise data collected from the physiological effects observed from interactions between cells from different species and a compound of interest. By examining the differences in the observed physiological effects, discovery of diagnostics and therapeutics useful in veterinary medicine may be developed.

A profile may comprise data collected from the physiological effects observed after interaction with a compound and cells expressing taste or smell receptors. This configuration may be desired when screening for an interaction that will produce a pleasant taste, an undesirable taste, or no taste. A profile that demonstrates taste receptor responses may be utilized to alter the taste of products such as foods and medicines.

A profile may comprise data collected from the physiological effects observed between a compound and cells collected during the aging process. In particular, cells may be collected during childhood, adolescence, and adulthood to examine changes in the physiological effects of a compound on each of these cells. These interactions may be useful in developing therapeutics that reduce the effects of aging.

A profile may comprise data collected from the physiological effects observed between a compound and cells collected during differentiation of stem cells. Stem cells can be treated with agents that induce differentiation. Examples of such agents are small molecules such as retinoic acid or DMSO, protein agents such as bone morphogenetic proteins or c-kit, or exogenously introduced genes such as HNF-1 or Oct 3/4. Cells may be sampled at various times after treatment and introduced into a flow cytometer. Cells can be monitored for differentiation-related markers such as N-Cam for neuronal cells and simultaneously tested for functional responses as described above. Data may be gathered as a function of time for a given treatment, or as a function of both time and treatment for multiple treatments. This approach is particularly useful for generating profiles of compounds in cell types that are difficult to culture, e.g., neurons, adipocytes, cardiac myocytes or chondrocytes. It is also useful for monitoring the effects of compounds on the differentiation process itself, thereby identifying compounds that may be toxic to developing embryos. Similarly, a profile may comprise data collected from the physiological effects observed between a compound and cells undergoing division versus cells that are growth arrested. Preferably, growth arrest is accomplished by plating the cells at a sufficiently high density to induce contact inhibition of proliferation.

VII. Profile Analysis

Preferably, profile analyses are conducted by comparing known pharmaceutically important compound profiles to those profiles of the test compounds in a way that allows a determination of possible physiological function. Comparisons may be made based on biological activity levels, the amount of a compound necessary to initiate an equal response, proteomic, or genomic expression levels. The term “proteomic expression” as used herein refers to a profile of expressed proteins in a cell based on their isoelectric point (“PI”) and size. Preferably these comparisons are made utilizing a computer program.

Results of profile comparisons may be displayed in the form of tables, graphs, or images. Tables may be in the form of spreadsheets listing raw data as well as any activity values calculated by comparisons. Examples of typical tables displaying profile comparisons are shown in the “examples” section below.

Preferably the tables may be transferred into data analysis software such as Excel™ so that statistics may be computed. Commercial visualization software such as Spotfire™ (Spotfire Inc, Somerville Mass.) may be used for profile comparisons. One may also use statistical methods, such as factor analysis, wherein correlations between control and test compounds are analyzed to reveal underlying relationships, cluster analysis, wherein a library of compounds may be sorted into groups based on similarities between their profiles, or time series analysis, and wherein time-dependent effects of compounds such as for example, effects after induction of stem cell differentiation are analyzed.

Database Construction

Data regarding the species origin and tissue from which the cells were derived, the nature of cells (tumor, metastatic, hybridoma, etc.), the passage number of the cells, their differentiation state, their gene expression profile, their functional responses to compounds, and standards may be archived in a database. Biological data are preferably linked to chemical structures and multivariate analysis techniques may be used to identify activity trends and correlate them with expressed genes. Patterns of biological activity correlated with gene expression patterns and with chemical structures may be used to select compounds for progression and to construct predictive models.

Preferably it would be beneficial to create a database or data structure in computer readable form utilizing DNA array technology, sequencing, PCR assays, microarray analyses, individual cell analyses, or any other suitable means. This data structure includes information regarding the particular genes of interest that are expressed in any particular cell. Thus, for example, a polynucleotide obtained directly or indirectly (e.g., cDNA) from each cell can be screened to create a qualitative or quantitative profile of the genes of interest expressed by that cell. If, for example, the genes of interest encoded GPCRs, the cells or a cDNA library from each of the cells could be screened against a microarray containing known or deduced GPCR DNA. A profile might then be generated for each cell, identifying the particular GPCR expression pattern for that cell type.

With DNA expression information in hand, the cells may be screened against compounds of interest. Through such screening, correlations may be made between the DNA expression patterns and the activity of a particular compound on particular cells.

In yet another embodiment of the invention, compounds known to have a pharmacological or biological activity of interest are screened against the library of cells, and the physiological responses of the cells to those compounds are measured and stored in the data structure. This can provide an empirical measurement of which cell types express genes involved in the physiological activity of each particular known compound, whether or not those genes are actually identified and characterized. Then, utilizing this knowledge base in the data structure, compounds of unknown activity can be screened against the same cells and the activity of those compounds in a desired assay may be measured. That measured activity can then be correlated with the activity of the known compounds, which will facilitate prediction of the pharmacological activity of the new compound. This embodiment of the invention can be used as a stand-alone technique, or more preferably it can be used in combination with any other that characterizes the DNA expression patterns of that particular cell.

The present invention may be used not only to determine the biological activity of a particular compound, but also to characterize the biological function of endogenous proteins. For example, in the course of the analysis described above, associations may in some circumstances be drawn between the activity of known and/or uncharacterized compounds and the expression of, for example, an orphan receptor. This correlation between expression of the orphan receptor and biological activity of compounds in cells that express that receptor may facilitate the assignment of a function to that receptor. Moreover, even if the physiological role of the orphan receptor is not fully ascertainable from the correlation, the information generated by the present invention may nonetheless provide a valuable tool for drug development by showing, e.g., an association between expression of that receptor and a desired pharmacological activity.

A database can be created comprising all of the information contained in the compound profiles as well as the information collected when preparing the assay configurations. Preferably, this information is stored and arranged in such a way that the information of one compound may be compared to the information of another compound as desired.

The database may also function as an inventory database collecting information about the cells used, the cells position within the assay apparatus, protocols necessary to perform the experiments, an inventory of reagents, and primer sequence information. The database may also contain chemical structures, either in two-dimensional or three-dimensional representations; derived structural descriptors; and gene or protein expression patterns (whether categorical (yes/no) or quantitative and whether target-related or result-related). Additionally, the database may contain externally generated data for all or a subset of the compounds, such as toxicity in vivo, ADME or DMPK data, efficacy in animal models, activity in other screening assays, physical parameters (such as solubility, stability, log P), and calculated data such as polar surface area. The database may further contain medical information linking specific genes or proteins with disease states, information regarding genetic knock out or overexpression of specific target genes, information on interactions between biological macromolecules or functional crosstalk between biological effectors, such as receptor crosstalk, three dimensional structures of proteins, representations of signal transduction pathways, data in image form such as histological sections, videos, photomicrographs or electron micrographs of cell morphology, quality control information such as purity of compounds, Z-factors for assays, activity of biological standards, cell background information such as tissue of origin, results of mycoplasma or virus testing, expression of lineage-specific marker genes, culture conditions and history, disease type, prior exposure to medications or toxic agents.

When the database contains information about the cells used in the experiments, the information may include identity of the cell, where the cell was obtained such as purchasing information, cell culture requirements, and references to journals utilizing the cell.

The database may be accessible through workstations and may be linked to additional databases such as GenBank or SwissProt through an Internet service provider. The database may have links to Internet service provider addresses such that the user may click on a link directing the computer to connect with the corresponding Internet address service provider. Some examples are one click links to the National Center for Biotechnology Information, “NCBI,” for Basic Local Alignment Search Tool (“BLAST”) analysis. The database may comprise email programs allowing the user to transmit results over the Internet.

The above disclosure generally describes the present invention. A more complete understanding can be obtained by reference to the following specific examples which are provided herein for purposes of illustration only and are not intended to limit the scope of the invention.

### EXAMPLE 1

### Isolation and Preparation of Cells and Cell Lines

The cell lines utilized in the present examples were purchased from commercial suppliers, such as ATCC. Isolation and preparation protocols were performed based on the suppliers recommendations.

### EXAMPLE 2

### Calcium Mobilization Assay Procedure

Changes in intracellular calcium levels and the mobilization of intracellular calcium are measured using a FLIPR (Molecular Devices Corporation, Sunnyvale, Calif.), a platform technology for high-throughput screening. The FLIPR system includes an argon laser, a PC, a 96-tip pipettor, a test chamber capable of holding multiple microtiter plates, and a CCD camera. An example of calcium measurement in the adherent cell line, ECV304, expressing the P2Y2 receptor (following the method of Sullivan, Calcium Signaling Protocols (Meth. Mol. Biol. 114:125-133, 1999) is described below.

Cells are plated at an appropriate density (for example, 1×10⁵ cells/well) in a transparent-bottomed, black-walled, 96-well plate and incubate overnight. Cells are loaded with 4 μm Fluo-3/acetoxymethyl (Fluo-3/AM) and washed 3× with wash buffer (typically PBS or HBSS, containing 20 mM HEPES, pH 7.3), then a volume of 100 μl of wash buffer is left in each well and place the plate in the test chamber. Plates are transferred to the FLIPR apparatus. To determine the laser output required for a mean fluorescent signal of approximately 12,000 fluorescent counts/well, the test plate is transferred to the test chamber and a “signal test” is initiated, in which the CCD camera captures an image of the test plate. The image is converted to a numerical fluorescence reading for each well.

A standard microtiter plate (addition plate 1) containing varying concentrations of suramin, a P2Y2 receptor antagonist, is placed in the right hand position of the FLIPR test chamber. Addition plate 2 containing varying concentrations of receptor agonist UTP is placed in the left hand position of the FLIPR test chamber. The laser power is set at 250 mW, the camera aperture at f2; and the camera exposure time at 0.4 seconds.

A 4× stock solution of suramin or UTP is prepared and added to wells containing the cells and 100 μl buffer, to equal a total of 200 μl per well. Readings (captured by a CCD camera and subsequently converted to digital data) are taken for 60 seconds at 1 second intervals.

### EXAMPLE 3

### Procedure for Measuring Changes in cAMP Levels

An enzyme immunoassay (EIA) or radioimmunoassay (RIA) can be used to determine cAMP concentration utilizing a cAMP-specific antibody. Commercially available assay kits may be used. Alternatively, cAMP-antibody immunocomplexes may be performed by a rapid filtration method using 96 well filtration plates (Millipore multiscreen, Bedford, Mass.). Initially, standard solutions of cAMP in a range of 0.0009 nM to 5 nM in 10 mM sodium acetate buffer are prepared. Similarly, sample extracts in 10 mM sodium acetate buffer, with varying dilutions are prepared. 50 μl cAMP standard or test sample are placed in each well and 25 μl of ¹²⁵I cAMP and 25 μl antiserum (diluted 1: 3,000) are added to each well. Plates are covered and incubated for 24 hours at 4° C. 50 μl antirabbit antibody coupled to a solid support (such as agarose or magnetic beads) are added to each well and the plate is vortexed. The plate is then incubated at 4° C. for 1 hour on a rotating platform. 100 μl of 12% PEG is then added to each well and the contents of the plate are filtered using vacuum manifold and washed twice with 200 μl PEG. Radioactivity in the immunoprecipitates contained in the wells is determined by counting on a γ-counter and the amount of cAMP present in the test samples is calculated using the standard curve generated by the cAMP standards.

### EXAMPLE 4

### Receptor Internalization Assay Procedure

Cellular receptors located at the plasma membrane are often internalized and recycled through the endocytic recycling compartment (ERC) upon a variety of events as agonist stimulation. The effect of various compounds on the internalization process can be measured using the ArrayScanII system (Cellomics, Pittsburgh, Pa.) a high-resolution imaging and analysis platform which has capabilities to quantify multiple fluorescently labeled cellular constituents. Commercially available screening kits (such as the HitKit, Cellomics, Pittsburgh, Pa.) contain optimized protocols and fluorescent reagents useful for this purpose.

The Receptor Internalization and Trafficking Application on the ArrayScanII can be used to determine compound dose-responsive curves, toxicicity and IC50 or EC50 values. The receptor Internalization and Trafficking Application can also quantify the association of other receptors with the ERC.

The ArrayScan II system may be used to study the internalization, recycling, and intracellular trafficking of the transferrin receptor (TfR) using the Receptor Internalization and trafficking application and the Transferrin Receptor HitKit following the method of Ghosh et al., Biotechniques 29:170-175, 2000.

COS-1 cells at a concentration of 10⁴ cells/well were placed in 96-well microplates in McCoy 5A medium with 5% fetal bovine serum and 4 μm deferoxamine mesylate (Sigma, St. Louis, Mo.) and incubated for 18 hours. The cells were washed 2× with Eagle's minimum essential medium (EMEM), subsequently incubated at 37° C. for 45 minutes with 20 μg/ml AxTf (Alexa 546 fluorophore conjugated to diferric transferrin), then rinsed 2× with PBS. The AxTf that accumulates in the cell appears as a bright red spot near the nucleus. The wells were fixed at 23C for 30 minutes with 100 μl/well of 3.7% formaldehyde solution. To assay for TfR recycling after AxTf incubation and removal, the wells were incubated at 37° C. for 60 minutes with EMEM containing 10% FBS. The cells were then rinsed with PBS and fixed as described above. Samples were analyzed by loading the multiwell plates onto the ArrayScanII System. The plate data was then exported to an Excel™ spreadsheet using software such as Cellomics data viewer software. Systems such as the ArrayScanII System are capable of automatically reading and analyzing the images collected from multiple channels. The use of multiple channels capable of quantifying fluorescence of varying wavelengths enables the quantitation of multiple targets in the same cell at the same time. For example, while measuring the transferrin internalization by the above method, cells can be quantitated by simultaneously measuring using the nuclear stain Hoechst 33342 dye to quantitate nuclei.

### EXAMPLE 5

### Morphological Assay Procedure

Alterations in cellular morphology in response to various types of stimuli or addition of compounds may be measured following the method of Kapur et al., Exp. Cell Res. 244:275-85, 1998, with adaptations to suit multiple assay requirements. The following example illustrates the use of a morphological assay to examine changes in cell shape, cell perimeter, and cytoskeletal changes using human umbilical vein endothelial cells (Clonetics, San Diego, Calif.). Cells are cultured in M-199 medium containing 2% FBS, endothelial cell growth factor (10 ng/ml), hydrocortisone (1 μg/ml), and 0.4% bovine brain extract at 37° C. in 5% CO₂ in an incubator. The cells are passed using trypsin-EDTA and used between seven and eight passages. The cells are then seeded into a 96-well plate. The compound of interest (or stimulus) is added and the cells are incubated for a period of time. Specific details of the experiment, such as the optimal incubation period, incubation temperature, concentration of the test compound to be added, etc. would need to be determined for each cell type and compound to be added. Cells are then digitally imaged and the data is processed using digital imaging analysis software such as Image Pro™ (MediaCybernetics, Silver Spring, Md.). Morphological measurements, such as cell perimeter, aspect ratio, and projected surface area may be determined using the image analysis software. Other cellular regions which may be present, such as cellular lamellipodia and migratory ruffles, may be measured.

Alterations in cytoskeletal components in response to a compound or stimulus may also be measured. For example, to examine changes in F-actin structure in response to the addition of a compound of interest, cells are subsequently washed in HBSS and fixed with 4% formaldehyde at 37° C. for 15 minutes. The cells are permeabilized with 0.2% Triton X (100) for 5 minutes at 4° C. and incubated with 10 units of rhodamine-labeled phalloidin (Molecular Probes Inc., Eugene, Oreg.) for 20 minutes. The fixed and stained cells are analyzed for cellular morphology and cytoskeletal organization using a laser scanning confocal microscopy in conjunction with the Image Analysis Software Image Pro™. The cytological and morphological changes in cells treated with test compounds or test stimuli are compared with untreated control cells to quantitate the alterations that have occurred. Statistical analysis of data from replicates within each experiment and between experiments is pooled and tested for normalcy and equality of variance. A one-way analysis of variance or two way analysis of variance and other statistical analyses are then performed.

### EXAMPLE 6

### Procedure for Measuring Secretion of Neurotransmitters

The following examples describe several methods of quantitating secretion of neurotransmitters, following the methods of Koert et al., J. Neurosci. 21:5597-606, 2001; Wang et al. Acta. Pharmacol. Sin. 21:623, 2000; and Tamura et al., J. Neurochem. 76:1153, 2001.

Immunocytochemistry to visualize serotonin: The visualization of serotonin in the cerebral and buccal ganglia of lymnaea stagnalis was performed as follows. Previously isolated cerebral and buccal ganglia were incubated in 0.5% type XIV protease (Sigma, St. Louis, Mo.) for 30 minutes and fixed overnight at 4° C. in a 1% paraformaldehyde/1% acetic acid solution. The cells were rinsed in 50 mM Tris-HCL, pH 7.6, 150 mM NaCl, and 2% Triton X-100 for 8 hours, then incubated overnight in anti-serotonin primary antibody diluted 1:250 in the above buffer. The cells were rinsed in the above buffer without Triton X-100 for 6 hours, then incubated overnight in fluorescein-labeled swine-antirabbit secondary antibody diluted 1:50. The material was rinsed in 50 mM Tris-HCL, pH 7.6, 150 mM NaCl for 4 hours, mounted in 1% ethylenediamine in 75% glycerol, and viewed with a Zeiss LSM 401 inverted laser scanning microscope (Zeiss, Jena, Germany).

Mass Spectrometry: Cell samples were transferred into 1 μl of matrix solution (10 mg of 2.5-dihydroxybenzoic acid dissolved in 1 ml of 7.5 mM trifluoroacetic acid in 30% acetonitrile. After the sample was dried, the material was placed in a matrix assisted laser desorption ionization mass spectrometer (MALDI-MS) for analysis.

Peptide analysis: Cell samples were boiled in 0.1M acetic acid for 8 minutes, centrifuged for 10 minutes at 4° C. The supernatant was separated on a reverse phase HPLC system using a 5 μm Nucleosil 250×2.1 mm C18 column (Hichrom, Reading, UK). Solvent A was 7.5 mM trifluoroacetic acid; Solvent B was 7.0 mM trifluoroacetic acid in 60% acetonitrile. The peptides were separated using a gradient from 5% solvent B to 100% solvent B with a flow rate of 300 μl/minute. Fractions were collected, and 0.5 μl of each fraction was submitted to MALDI-MS (see above) for further analysis. Selected fractions were also submitted for amino acid analysis using an ABI 432A peptide synthesizer (Applied Biosystems, Foster City, Calif.).

Preparation of synaptosomes for glutamine, GABA, or serotonin release assays: Rat brain synaptosomes were prepared following the detailed procedure of Tamura et al., J. Neurochem. 76:1153, 2001, which is incorporated herein by reference. Briefly, 1.5 g of rat cerebrum was homogenized in 10 ml of ice-cold 0.32 M sucrose in 4 mM Tris-HCL, pH 7.4 (sucrose solution) with a glass-Teflon homogenizer. The synaptosomes were isolated and washed by centrifugation as described in the text. The resulting pellet was used as the crude synaptosome preparation. The synaptosomes were then frozen/thawed following Nicholls et al., J. Neurochem. 52:331, 1989. Synaptosomes were then resuspended in 200 μl of solution A: (140 mM K⁺ gluconate; 4 mM KCl; 4 mM MgSO₄; 2 mM Tris-ATP; 20 mM HEPES; 50 μM glutamate, pH 7.4) at 3.75 mg/ml. 50 μl of (300 mM NaCl in 20 mM Tris-HCl buffer, pH 7.4) was added. 117.5 μl of solution A was added, followed by 7.5 μl of [³H]glutamate (50 Ci/mmol, Amersham Pharmacia Biotech, Piscataway, N.J., USA). An equal volume of 10% DMSO was added. The entire mixture was frozen/thawed, and subsequently treated as described in Tamura (supra).

Assay for glutamate release: The washed synaptosomes loaded with [³H]glutamate were preincubated at 30° C. for 1.5 minutes. Aliquots were incubated with various release solutions for various time periods at 30° C. as detailed in the text. The release reaction was stopped by placing the samples on ice, followed by centrifugation at 10,000 g for 10 minutes at 4° C. Radioactivity in the supernatant was determined using a Beckman LS 6500 scintillation spectrophotometer.

Assay for GABA and serotonin release: The assays for GABA and serotonin release were similar to that described for the glutamate release assay, except for the following: the ³H glutamate was replaced with 7.5 μCi of 4-amino-n-[2,3-³H] butyric acid or 5 hydroxy[G-³H]tryptamine, creatine sulfate, and the 50 μM glutamate was replaced with 50 μM GABA or 50 μM serotonin (Amersham Pharmacia Biotech, Piscataway, N.J., USA).

### EXAMPLE 7

### Procedure for Measuring Membrane Potential

Cells were plated in 96 well black well clear bottom tissue culture-treated plates (Coming Inc, Corning, N.Y.) at a density of 10,000 to 100,000 cells/well in 100 ul of culture medium and incubated for 18 hours. Membrane potential assay reagent 100 ul (Molecular Devices, Sunnyvale, Calif.) was added and the plate incubated at 37° C. for 1 hour. Compounds were added to the cells and changes in fluorescence monitored for 5 minutes using a FLIPR instrument (Molecular Devices, Sunnyvale, Calif.).

### EXAMPLE 8

### Procedure for Determining Light Scattering Properties

Compounds were added from 10 mM DMSO stocks at the desired final concentration and incubated for 30 minutes to 24 hours. Cells were detached from culture vessels using trypsin EDTA solution (Mediatech, Inc, Herndon Va.) and resuspended in PBS buffer (Mediatech, Inc, Herndon Va.). The cell suspension was aspirated into a flow cytometer equipped with a 488 nM argon laser (Cytomation, Fort Collins, Colo.) and forward and side scatter properties were measured as recommended by the instrument vendor.

### EXAMPLE 9

### Cellular Apoptosis Assay Procedure

Cells were plated in 96 well culture-treated plates (Corning Inc, Corning, N.Y.) at a density of 20,000 to 100,000 cells/well in 100 ul of culture medium and incubated for 18 hours. Compounds were added to the cells and incubated at room temperature for 3 to 6 hours. Cells were washed 1 times with PBS (Mediatech, Inc, Herndon Va.) and Caspase 3 activity in cell extracts was measured using the EnzChek® Caspase-3 Assay Kit (Molecular Devices, Sunnyvale Calif.) as described by the manufacturer.

### EXAMPLE 10

### Mitochodrial Membrane Potential Assay Procedure

Cells were plated in 96 well black well clear bottom tissue culture-treated plates (Coming Inc, Corning, N.Y.) at a density of 10,000 to 100,000 cells/well in 100 ul of culture medium and incubated for 18 hours. Compounds were added from 10 mM DMSO stocks at the desired final concentration and incubated for 30-240 minutes. Mitochondrial membrane potential was measured as described in Current Protocols in Cytometry (Wiley) Chapter 7, using a MoFlo flow cytometer equipped with a 488 nm argon laser (Cytomation, Fort Collins, Colo.).

### EXAMPLE 11

### Procedure for Determining Membrane Integrity

Cells were detached from culture vessels using trypsin-EDTA solution and resuspended in PBS buffer (Mediatech, Inc, Herndon Va.) at 5 million cells/ml. Compounds were added to the cells and incubated at 37° C. for 4-24 hours. Propidium Iodide (Molecular Probes, Eugene Oreg.) was added to the cell suspension to 0.5 mg/ml. Live/dead discrimination was determined by measuring fluorescence excitation using a MoFlo flow cytometer equipped with a 488 nm argon laser (Cytomation, Fort Collins, Colo.).

### EXAMPLE 12

### A Data Set Compilation and Comparison to Identify a Potential Candidate Compound That May Act as a Target Molecule Modulator

The cells shown below were obtained from the American Type Culture Collection (ATCC), P.O. Box 1549 Manassas, Va. 20108 and cultured according to the recommendations of the supplier.

TABLE 1
Cell lines used in the present examples.

| Cell line | Tissue | Cells/well |
|---|---|---|
| T-47D | epidermal (breast) | 75,000 |
| NCI-H460 | Tracheobronchial (lung) | 60,000 |
| SaOS-2 | Musculoskeletal (bone) | 20,000 |
| PANC-1 | Endocrine (pancreas) | 50,000 |
| PC-3 | Urogenital (prostate) | 20,000 |
| NTera-2 | Stem cell (neural progenitor) | 30,000 |
| Jurkat | Blood (T-cell) | 100,000 |
| COLO320 | Digestive (colon) | 120,000 |
| HBL-100 | epidermal (breast) | 50,000 |
| SK-Hep-1 | Circulatory (endothelial) | 50,000 |

Table 1 shows the cell lines used in the present example, the tissues from which they were derived and the plating densities used in assays.

The above panel of 10 cell lines was plated in 96-well black, clear-bottomed tissue culture-treated plates (Molecular Devices, Sunnyvale, Calif. with the number of cells per well indicated in Table 1. Cells were maintained overnight at 37° C., 5%CO₂ in a humidified incubator. Media was removed and the cells were then incubated in Hanks balanced salt solution (Invitrogen, Carlsbad Calif.)/10 mM HEPES pH 7.4 containing 0.04% Pluronic Acid (Molecular Probes, Eugene Oreg.) and 4 uM Fluor-3AM (Molecular Probes, Eugene Oreg.) for one hour at 37° C.

Antagonists were added to the indicated concentration and the cells incubated at room temperature for 20 minutes. After 10 seconds of baseline read collection, agonists were added on a FLIPR instrument (Molecular Devices, Sunnyvale, Calif.) and data were collected for a total of 3.5 minutes. The fluorescence emission was normalized to initial intensity and the sum of the signal over the entire time period was exported and used for determining percent of control responses using the following formula: Percent of Control=(Sample-unstimulated cells)/(ionomycin treated cells-unstimulated cells). The following treatments were used: buffer (Hanks balanced salt solution, Invitrogen, Carlsbad Calif.), (low control), 10 uM ionomycin (Calbiochem San Diego Calif.), (high control), 10 uM N-alphamethyl histamine (Tocris Ellisville Mo., Compound 1), 10 uM R-alphamethyl histamine (Sigma, St. Louis, Mo., Compound 2) and two known agonists: 10 uM histamine (Sigma, St. Louis, Mo.)and 1.0 uM bradykinin (Calbiochem, San Diego, Calif.). In the antagonist example, final concentrations in uM were: histamine 10, chlorpheniramine (Sigma, St Louis, Mo.) 10, terfenadine (Sigma, St Louis, Mo.) 1 and 10, bradykinin 1, ionomycin 10. Calcium mobilization was measured as described above. Histamine H1 receptor expression was determined by RT-PCR as described below.

The agonist example data (Table 1) show that Compound 1 produces a similar pattern of responses to that of histamine and a different pattern from that of bradykinin, whereas Compound 2 resembles neither bradykinin nor histamine. Thus Compound 1 is identified as having similar physiological effects as the pharmacologically important mediator, histamine. Neither Compound 1 nor histamine were active in cells that do not express the H1 receptor target, but not all cells that express the target are responsive to histamine or Compound 1.

TABLE 2
(agonist example).

| | T-47D | NCI-H460 | SaOS-2 | PANC-1 | PC-3 | NTera-2 | Jurkat | COLO320 | HBL-100 | SK-He1 |
|---|---|---|---|---|---|---|---|---|---|---|
| histamine_buffer | 0 | 32 | 13 | 26 | 1 | 0 | 0 | 1 | 18 | 19 |
| BK_Buffer | 0 | 0 | 22 | 1 | 0 | 1 | 1 | 1 | 9 | 0 |
| N-a-his_Buffer | 0 | 30 | 11 | 22 | 1 | 2 | 2 | 1 | 16 | 12 |
| R-a-his_Buffer | 0 | 0 | 0 | 0 | 0 | 2 | 2 | 1 | −1 | −1 |
| ionomycin_buffer | 100 | 100 | 100 | 100 | 100 | 100 | 100 | 100 | 100 | 100 |
| buffer control | 0 | 0 | 0 | 0 | 0 | 0 | 0 | 0 | 0 | 0 |
| H1R expression | N | Y | Y | Y | Y | N | Y | Y | Y | Y |

Table 2 (above) shows a data structure (profile) of the results of testing two agonist-like compounds in 10 cell lines and a comparison with the activities of two known agonists, histamine and bradykinin. A non-specific calcium activator (ionomycin) is included as a positive control. Expression of the target receptor, histamine subtype H1 is also part of the profile.

The antagonist example shows testing of Compound 1 (terfenadine) at 1 or 10 uM for its effects on histamine and bradykinin-induced calcium mobilization in the above panel of 10 cell lines. This compound inhibited calcium responses to histamine but at 1 uM it did not inhibit responses to bradykinin, suggesting H1 antagonist activity. However, at 10 uM, Compound 1 partially inhibited responses to bradykinin in SAOS-2 cells, indicating non-specific effects. Compound 2 (chlorpheniramine), a known H1 antagonist, was tested in the same assay and had similar effects to Compound 1, but did not inhibit bradykinin responses at 10 uM. Thus Compound 1 was identified as having similar physiological effects to a known H1 antagonist, but was also identified as less specific in its action.

TABLE 3
Antagonist example

| | Jurkat | COLO32ODM | NCI-H460 | PANG-1 | SaOS-2 | T-47D | NTera-2 | HBL-1 |
|---|---|---|---|---|---|---|---|---|
| histamine_buffer | −2 | 0 | 24 | 30 | 18 | 2 | 0 | 27 |
| Histamine_C1PhAm | 8 | 0 | −2 | −1 | 0 | 2 | 0 | 0 |
| Histamine_terfenadine 10 | 4 | 0 | 1 | 0 | −1 | 0 | 0 | −1 |
| Histamine, terfenadine 1 | 4 | 0 | 3 | 15 | 0 | 2 | 0 | 0 |
| BK_Buffer | 6 | 0 | −1 | −2 | 29 | 2 | 0 | 12 |
| BK_C1PhAm | 12 | 0 | −1 | 1 | 29 | 2 | 0 | 11 |
| BK_terfenadine 10 | 8 | 1 | 1 | 1 | 4 | 0 | 0 | 11 |
| BK, terfenadine 1 | 6 | 1 | −2 | 1 | 32 | 2 | 0 | 16 |
| ionomycin_buffer | 100 | 100 | 100 | 100 | 100 | 100 | 100 | 100 |
| ionomycin_C1PhAm | 70 | 98 | 73 | 101 | 96 | 102 | 99 | 87 |
| ionomycin_terfenadine | 193 | 98 | 83 | 96 | 97 | 110 | 100 | 88 |
| buffer control | 0 | 0 | 0 | 0 | 0 | 0 | 0 | 0 |

Table 3 shows a data structure (profile) of the results of testing an antagonist-like compound (terfenadine) in 10 cell lines and a comparison with the activities of a known H1 antagonist, chlorpheniramine. The compounds are assessed against two agonists, histamine and bradykinin and against a non-specific calcium activator (ionomycin).

### EXAMPLE 13

### Identification of a Potential Candidate Compound with Similar Efficacy as a Known Target Molecule Modulator

Compound 1 (chlorpheniramine, 10 uM), Compound 2 (terfenadine, 10 uM) and compound 3 (U73122, Sigma, St Louis, Mo., 10 uM) were assayed for their effects on histamine-induced calcium mobilization in a panel of 10 cell lines as described in Example 2. Expression of the target H1 receptor was measured by RT-PCR of mRNA samples from each cell line, as follows.

Protocol for mRNA Isolation from Dynabeads:

Cells were harvested in log phase growth the day following a media change and stored at—−80° C. The cell pellet was removed from freezer and resuspended in 2 ml Cell Lysis Buffer (100 mM Tris-HCL pH 7.5, 500 mM LiCl, 10 mM EDTA, 1% lithium dodecyl sulfate 5 mM dithiothreitol (Dynal Biotech Inc. Lake Success, N.Y.) per 1×107 cells. The cells were kept on ice.

Dynabeads (Dynal Biotech Inc. Lake Success, N.Y.) were mixed and 500 μl was removed for each isolation to a fresh 1.5 ml tube. The dynabeads were rinsed with equal volume of Cell Lysis Buffer and then the buffer was removed. 1 ml of cell lysate was added to the beads and the beads were resuspended and placed on a rotator for 5′ at room temperature. The lysate was discarded and the dynabeads were rinsed twice with 1 ml Wash Buffer A (10 mM Tris-HCl pH 7.5 0.15 M LiCl, 1 mM EDTA 0.1% lithium dodecyl sulfate).

The dynabeads were rinsed ×2 with 1 ml Wash Buffer B (10 mM Tris-HCl, pH 7.5, 0.15 M LiCl 1 mM EDTA) while being transferred to a clean 1.5 ml tube. The dynabeads were quickly rinsed once with 1 ml ice-cold 10 mM Tris HCl (pH 7.5), then the wash was removed. 90 μl of 10 mM Tris Tris HCl (pH 7.5) was added and the beads were incubate at 70-80° C. for 10 minutes. The eluted mRNA was removed to a clean tube on ice. 1 ml of cell lysate was added to the beads and the beads were resuspended. The above process was repeated for a total of five elutions (450 ul of mRNA).

The dynabeads were added with 10 U DNAse I (Invitrogen, Carlsbad Calif.), 50 ul 10× Dnase I buffer (200 mM Tris-HCl pH 8.4, 20 mM MgCl2, 500 mM KCl) at RT for 15 minutes. 2 μl of 0.5 M EDTA pH 8.0 was added and the DNAse was heat inactivated at 80° C. for 10 minutes. The sample was then transferred to 4° C. and cDNA synthesis proceeded.

CDNA Synthesis

CDNA Synthesis was accomplished by splitting the mRNA into two tubes of 250 μl and set one tube aside as the negative control. To the other tube, 25 μl 3 μg/μl Random Primer oligonucleotides (Invitrogen, Carlsbad Calif.) and 25 μl 10 mM dNTP mix (10 mM each dATP, DTTP, dGTP, dCTP) (Applied Biosystems, Foster City Calif.) were added. The material was incubated for 10 minutes at 70-80° C., moved to ice, and spun briefly.

100 ml of 5× First Strand Buffer (250 mM Tris-HCl pH 8.3)(Invitrogen, Carlsbad Calif.) as added, followed by 50 μl of 100 mM DTT (Invitrogen, Carlsbad Calif.) and 25 μl of RNAse inhibitor (Promega, Madison Wis.). The contents were mixed and incubated at room temperature for 10 minutes.

25 μl SuperScript II (Invitrogen, Carlsbad Calif.) were added and the contents of the tubes were mixed by pipetting. The tubes were then incubated at 42° C. for 50′. The reaction was stopped by heating the contents of the tubes at 70° C. for 15 minutes.

450 μl 10 mM Tris (pH 7.5) were added to 10 tubes and then 50 μl cDNA from the tubes containing SuperScript II which had been incubated and heated were added to each tube. The contents were aliquotted and frozen at −80° C. 10 μl of cDNA and negative control were aliquotted from each cell line to duplicate wells of a 96-well PCR plate (Applied Biosystems, Foster City Calif.)).

PCR:

The following primer sets were used for PCR:

| | |
|---|---|
| 5HT4U | 5′-TCCTCTGGCTCGGCTAT |
| 5HT4L | 5′-TGTATGGGCAGTTTCTCGAGT |
| B-actinU | 5′-CACCACACCTTCTACAATGAGCTG |
| B-actinL | 5′-AGGATCTTCATGAGGTAGTC |
| H1HRU | 5′-TACAAGGCCGTACGACAACAC |
| H1HRL | 5′-GGTTGACGGCTACATAGTCCC |
| H2HRU | 5′-AAGTGGAGCTTTGGCAAGGTC |
| H2HRL | 5′-CACATGATCAGTAGCGGGAGG |
| H3HRU | 5′-CTGTGGCTGGTAGTGGACTAC |
| H3HRL | 5′-TGTAGAAGAACTCGGCATAGC |
| H4HRU | 5′-TACATCCCTCACACGCTGTTC |
| H4HRL | 5′-TTGGCCCATTCACTAAGAAGG |

Each 25 μl reaction contains 10 μl cDNA or no reverse transcriptase control, 0.625 U Taq Gold (Applied Biosystems, Foster City Calif.), 2 mM MgCl₂, 2.5 μL 10× PCR II buffer (Applied Biosystems, Foster City Calif.), 50 μM each dATP, DTTP, dGTP, dCTP and 200 nM PCR primers. Reactions were incubated at 92° C. 8 minutes and cycled 30 times 94° C. 30 seconds, 55° C. 30 seconds, 72° C. 30 seconds. The entire reaction was loaded onto a 20×20 cm 1% TAE gel, run at 150 volts for 4 hours, stained with Syber Gold (Molecular Probes, Eugene Oreg.) 30 minutes and photographed.

The data show that terfenadine and U73122 partially inhibit the response to ionomycin in T-47D and Ntera-2 cells, neither of which express the HI receptor. By contrast, chlorpheniramine does not affect the ionomycin response in either cell line. Furthermore, U73122 inhibits the responses of SaOs-2 and HBL-100 to bradykinin, which does not activate the target H1 receptor, whereas chlorpheniramine does not. Thus although terfenadine and U73122 inhibit responses to histamine, they are identified as less specific than chlorpheniramine as inhibitors of histamine signaling through the H1 receptor.

TABLE 4
Specificity analysis of three compounds in a panel of 10 cell lines.

| | T-47D | NCI-H460 | SaOS-2 | PANC-1 | PC-3 | NTera-2 | Jurkat | COLO320 | H |
|---|---|---|---|---|---|---|---|---|---|
| histamine_buffer | 0 | 32 | 13 | 26 | 1 | 0 | 0 | 1 | |
| Histamine_C1PhAm | 0 | 0 | −1 | 0 | 0 | 0 | 5 | 1 | |
| Histamine_terfenadine 10 | 0 | 1 | −1 | 0 | 0 | 0 | 4 | 0 | |
| Histamine_U73122 | 1 | 2 | 0 | 2 | 0 | 2 | 2 | 2 | |
| ionomycin_buffer | 100 | 100 | 100 | 100 | 100 | 100 | 100 | 100 | |
| ionomycin_C1PhAm | 102 | 96 | 112 | 104 | 104 | 119 | 123 | 95 | |
| ionomycin_terfenadine 10 | 78 | 86 | 48 | 90 | 88 | 74 | 79 | 79 | |
| ionomycin_U73122 | 88 | 102 | 71 | 89 | 100 | 72 | 52 | 73 | |
| BK_Buffer | 0 | 0 | 22 | 1 | 0 | 1 | 1 | 1 | |
| BK_C1PhAm | 0 | 0 | 23 | 0 | 0 | 2 | 10 | 0 | |
| BK_U73122 | 1 | 1 | 2 | 0 | 0 | 1 | 52 | 5 | |
| BK_terfenadine 10 | 0 | 1 | 4 | 1 | 0 | 0 | 8 | 1 | |
| buffer control | 0 | 0 | 0 | 0 | 0 | 0 | 0 | 0 | |
| H1R expression | N | Y | Y | Y | Y | N | Y | Y | Y |

Table 4 shows a data structure (profile) of the results of a specificity analysis of two antagonist-like compounds, terfenadine and U73122, in 10 cell lines and a comparison with the activities of a known H1 antagonist, chlorpheniramine. Expression of the target receptor, histamine subtype H1 is also part of the profile. The compounds are assessed against two agonists, histamine and bradykinin and against a non-specific calcium activator (ionomycin).

What is claimed is:

1. A method for ascertaining the functional patterns of pharmacologically-important compounds by measuring the physiological effect of a plurality of compounds on a plurality of cells, comprising the steps of:
a. assaying a plurality of compounds to obtain a first set of data reflecting the physiological effect of each compound of said plurality of compounds on each cell of said plurality of cells;
b. producing a second set of data reflecting the physiological effect of at least one known pharmaceutically-important compound; and
c. comparing said first and second sets of data to identify a compound of said plurality of compounds having similarities to or differences from said at least one known pharmaceutically-important compound, thereby ascertaining said functional patterns of said identified compound.

2. A method for inferring the biological activity of an uncharacterized compound by determining its physiological effect in a plurality of cells comprising the steps of:
a. assaying said uncharacterized compound to obtain a first set of data reflecting said physiological effect of said uncharacterized compound on each cell of said plurality of cells;
b. providing a second set of data reflecting the physiological effect of said at least one compound of known biological activity on each cell of said plurality of cells; and
c. comparing said first and second sets of data to determine similarities or differences between the physiological effects of said uncharacterized compound and said at least one compound of known biological activity, thereby inferring said biological activity of said uncharacterized compound.

3. A method for selecting a compound, from a plurality of compounds, that has specificity for a target molecule, comprising the steps of:
a. assaying said plurality of compounds to obtain a first set of data reflecting the physiological effect of each compound of said plurality of compounds on each cell of said plurality of cells;
b. providing a second set of data reflecting which cells of said plurality of cells expresses said target molecule; and
c. comparing said first and second sets of data to select a compound from said plurality of compounds that exhibits minimal effects on said cells of said plurality of cells that do not express the target molecule.

4. A method for identifying a compound, from a plurality of compounds, that modulates a target molecule, comprising the steps of:
a. assaying said plurality of compounds to obtain a first set of data reflecting the physiological effect of each compound of said plurality of compounds on each cell of a plurality of cells;
b. providing a second set of data reflecting which cells of said plurality of cells express or do not express said target molecule;
c . assaying at least one known target molecule modulator to obtain a third set of data reflecting the physiological effect of said known target molecule modulator on each cell of said plurality of cells; and
d. comparing said first, second and third sets of data to identify a compound having similar physiological effects as said known target molecule modulator thereby selecting a compound having improved selectivity for said target molecule.

5. A method according to claim 4, wherein said plurality of cells are selected from the group consisting of endothelial cells, connecting tissue cells, epidermal cells, hematopoietic cells, stem cells, differentiated daughter cells derived from stem cells, central nervous system cells, endocrine cells, tracheobronchiolar cells, muscle cells, urogenital cells and digestive tract cells.

6. A method according to claim 5 wherein said endothelial cells are atrial endothelial cells or vascular endothelial cells.

7. A method according to claim 5 wherein said connective tissue cells are selected from the group consisting of osteoblast cells, osteoclast cells, chondrocyte cells synoviocyte cells, fibrosarcoma cells and osteocyte cells.

8. A method according to claim 5 wherein said epidermal cells are selected from the group consisting of melanocyte cells, keratinocyte cells, skin fibroblast cells, mammary ductal cells, mammary epithelial cells, corneal epithelial cells, hair follicle cells, papilla cells and submaxillary gland cells.

9. A method according to claim 5 wherein said hematopoietic cells are selected from the group consisting of lymphoblast cells, monocyte cells, T-cells, B-cells, neutrophil cells, eosinophil cells, erythroblast cells, granulocyte cells and dendritic cells.

10. A method according to claim 5 wherein said stem cells are selected from the group consisting of embryonic stem cells, teratocarcinoma cells, neural precursor cells and bone marrow stem cells.

11. A method according to claim 5 wherein said central nervous system cells are selected from the group consisting of astrocyte cells, ganglionic cells, cerebellum cells, neuroblast cells and neuronal differentiated cells.

12. A method according to claim 5 wherein said endocrine cells are selected from the group pancreas cells, thyroid cells, pituitary cells, and adrenal cells.

13. A method according to claim 5 wherein said tracheobrochial cells are selected from the group consisting of lung cells, tracheal cells and bronchiolar epithelium cells.

14. A method according to claim 5 wherein said muscle cells are smooth muscle cells, striated muscle cells or cardiac muscle cells.

15. A method according to claim 5 wherein said urogenital cells are selected from the group consisting of kidney epithelium cells, kidney mesangium cells, bladder cells, ovary cells, uterus cells, testis cells, placenta cells and prostate cells.

16. A method according to claim 5 wherein said digestive tract cells are selected from the group consisting of liver cells, stomach cells, intestine cells, gall bladder cells and esophagus cells.

17. A method according to claim 4 wherein said plurality of cells are healthy cells, diseased cells, or a combination of healthy and diseased cells.

18. A method according to claim 17 wherein said diseased cells are cells associated with a medical condition wherein said medical condition is selected from the group consisting of an infectious disease, cancer, an immune disease, a central nervous system disorder, cardiovascular disease, metabolic disorder, a musculoskeletal disorder, an epidermal disorder, a reproductive disorder and aging.

19. A method according to claim 18 wherein said infections disease cells are selected from the group consisting of virally infected cells, bacterially infected cells, fungally infected cells, protozoally infected cells and mycobacterial infected cells.

20. A method according to claim 18 wherein said cancer cells are selected from the group consisting of carcinoma cells, sarcoma cells, mesothelioma cells, leukemia cells, melanoma cells, papilloma cells, glioblastoma cells, astroctyoma cells, neuroblastoma cells and metastatic tumor cells.

21. A method according to claim 18 wherien said immune disease cells are selected from the group consisting of automimmune disease cells, allergic disease cells, inflammatory disease cells and immunodeficiency disease cells.

22. A method according to claim 18 wherein said central nervous system disorder is selected from the group consisting of a psychiatric disorder, a neurogeneretive disorder, a neruoinflammatory disorder, an effective disorder and a stroke.

23. A method according to claim 18 wherein said cardiovascular disease is selected from the group consisting of hypertension, atherosclerosis, myocardial infarction, ventricular hypertrophy, cardiac arrhythmias, congestive heart failure and pulmonary hypertension.

24. A method according to claim 18 wherein said metabolic disorder is diabetes or obesity.

25. A method according to claim 18 wherein said medical condition is a musculoskeltal disorder selected from the group consisting ostoarthritis, rheumatoid arthritis, osteoporosis and myasthenias.

26. A method according to claim 18 wherein epidermal disorder is psoriasis, dermatitis or alopecia.

27. A method according to claim 18 wherein said reproductive disorder is erectile dysfunction or infertility.

28. A method according to claim 20 wherein said carcinoma cells are selected from the group consisting of breast carcinoma cells, prostate carcinoma cells, ovarian carcinoma cells, non-small cell lung carcinoma cells, colorectoal carcinoma cells and esophageal carcinoma cells.

29. A method according to claim 19 wherein said virally infected cells are selected from the group consisting of cells infected with human immunodeficiency virus, cytomegalovirus, respiratory syncytial virus, rhinovirus, rotovirus, influenza virus, hantavirus and ebola virus.

30. A method according to claim 17 wherein said plurality of cells are a combination of healthy and diseased cells wherein said cells are of the same histological origin.

31. A method according to claim 17 wherein said plurality of cells are a combination of healthy and diseased cells wherein said cells are subclones of a parental cell.

32. A method according to claim 17 wherein said plurality of cells are a combination of healthy and diseased cells wherein said cells are differentiated cells from a precursor cell population.

33. A method according to claim 17 wherein said plurality of cells are a combination of healthy and diseased cells wherein said cells are from a common tissue.

34. A method according to claim 4 wherein said physiological effects are determined by assays for cellular membrane potential, intercellular calcium levels and cAMP levels.

35. A method according to claim 4 wherein said physiological effects are determined by assays selected from the group consisting of an optical assay, a gene expression assay, a phenomenological assay, a physiological transport assay, a cell proliferation assay, a physiological secretion assay, an apoptosis assay, and a toxicity assay.

36. A method according to claim 35 wherein said optical assay is a light scattering assay.

37. A method according to claim 35 wherein said gene expression assay is an assay to determine the production of disease-specific mRNAs or changes in cell surface markers.

38. A method according to claim 35 wherein said phenomenological assay is selected from the group consisting of a morphology change assay, a temperature sensitivity assay, a motility assay, a syncytia formation assay, a chemotaxis assay and an adhesion assay.

39. A method according to claim 35 wherein said physiological transport assay is a compound uptake assay or a compound efflux assay.

40. A method according to claim 35 wherein said cell proliferation assay is a DNA synthesis assay, an apoptosis assay or an anchorage-independent growth assay.

41. A method according to claim 35 wherein said physiological secretion assay is a cytokine production assay, a hormone secretion assay or a neurotransmitter secretion assay.

42. A method according to claim 35 wherein said toxicity assay is selected from the group consisting of a quantitative reactive oxygen species assay, an amyloid production assay, a mitochondrial membrane potential assay and a membrane integrity assay.

43. A data structure for ascertaining the functional patterns of a compound from a plurality of compounds comprising data obtained by determining the physiological effect of said compound on each cell of said plurality of cells and data obtained by determining the physiological effect of at least one known biologically active compound on each cell of said plurality of cells.

44. A data structure comprising a first set of data prepared according to the method of claim 4.

45. A data structure comprising a second set of data prepared according to the method of claim 4.

46. A data structure comprising a third set of data prepared according to the method of claim 4.

47. A compound identified using the method according to claim 4 wherein said compound modulates a target molecule in at least one cell of said plurality of cells.

48. A compound selected using the method according to claim 3.
